(19) United States
(12) Patent Application Publication
(10) Pub. No.: US 2010/0120042 A1
(43) Pub. Date: May 13, 2010

(54) METHOD OF SCREENING MATERIAL FOR IMPROVING SKIN FUNCTIONS

(75) Inventors: Minsoo NOH, Seoul (KR); Hyeonju Yeo, Seoul (KR); Seon Mi Park, Yongin-si (KR); Dong Wook Shin, Seoul (KR); Hyoung Ho Lee, Yongin-si (KR); Han-Kon Kim, Suwon-si (KR)

Correspondence Address: SUGHRUE MION, PLLC, 2100 PENNSYLVANIA AVENUE, N.W., SUITE 800, WASHINGTON, DC, 20037, US

(73) Assignee: Amorepacific Corporation, Seoul (KR)

(21) Appl. No.: 12/575,944

(22) Filed: Oct. 8, 2009

(30) Foreign Application Priority Data
Nov. 7, 2008 (KR) 10-2008-0110377

(51) Int. Cl. C12Q 1/68

(52) U.S. Cl. 435/6

(57) ABSTRACT

A method of screening a material for improving skin functions includes: (a) treating a skin cell with a candidate material; (b) detecting a change in a relative expression level of membrane-associated protein 17 (MAP17) gene; and (c) selecting a candidate material inducing the change in the expression level of the gene as a material for improving skin functions. That is to say, a material for improving skin functions is screened using MAP17 gene as a marker, on the basis of the change in the expression level of the MAP17 gene. A material for improving skin functions, which is useful in improving skin barrier function, promoting skin moisturization, preventing skin aging, or ameliorating skin troubles, may be effectively screened.

### CROSS-REFERENCE TO RELATED APPLICATION

This application claims priority to Korean Patent Application No. 2008-0110377, filed on Nov. 7, 2008, and all the benefits accruing therefrom under 35 U.S.C. §119, the contents of which in its entirety are herein incorporated by reference.

### BACKGROUND

1. Field

This application relates to a method of screening a material for improving skin functions.

2. Description of the Related Art

The epidermis is the outermost layer of the skin. When the stratum corneum, composed of keratinocytes, is in normal state, the epidermis acts as the body's major barrier against various stimulations and prevents the emission of moisture from the body.

The keratinocytes proliferate in the basal layer, the innermost skin layer, and differentiate gradually as they pass through the spinous layer and the granular layer. Through this keratinization process, the keratinocytes produce natural moisturizing factors (NMFs) and lipids (ceramides, cholesterols, and fatty acids), and form the stratum corneum, thereby providing the skin barrier function.

However, in case of skin diseases or troubles, the normal function of the stratum corneum is not maintained because of several reasons. As a result, the skin barrier is damaged, resulting in skin dryness and, in severe cases, inflammations.

In such skin disease-related inflammations, T helper 1 (Th1), T helper 2 (Th2) and T helper 17 (Th17) cells produce several interleukins, thereby inducing immune response.

Membrane-associated protein 17 (MAP17) is a 17 kDa sized membrane-associated protein. It was first observed that MAP17 gene was overexpressed in the renal carcinoma tissue, as compared with the normal renal parenchyma (Kocher et al., Clinical Cancer Research, Vol. 1:1209, 1995). It was thought that MAP17 protein is involved in hyperproliferation, which is characteristic of carcinoma tissue, since it was overexpressed in the carcinoma tissue. However, when MAP17 was overexpressed in the colon carcinoma cell line, it was verified that cell proliferation decreased under in vitro condition and tumor proliferation decreased under in vivo condition. Thus, it was revealed that MAP17 is irrelevant to cell proliferation (Kocher et al., Am. J. Pathol., Vol. 149:493, 1996). To conclude, the role of MAP17 is not known exactly as yet.

### SUMMARY

This disclosure is directed to providing a novel screening method capable of effectively screening a material for improving skin functions which improves skin barrier function, promotes skin moisturization, or prevents skin aging.

A method for screening a material for improving skin functions according to the disclosure includes: (a) treating a skin cell with a candidate material; (b) detecting a change in a relative expression level of membrane-associated protein 17 (MAP17) gene; and (c) selecting a candidate material inducing the change in the expression level of the gene as a material for improving skin functions.

This disclosure provides a novel screening method capable of effectively screening a material for improving skin functions which improves skin barrier function, promotes skin moisturization, or prevents skin aging. Thus screened material may be adequately used as an effective ingredient of a composition for improving skin functions.

### BRIEF DESCRIPTION OF THE DRAWINGS

The above and other aspects, features and advantages of the disclosed exemplary embodiments will be more apparent from the following detailed description taken in conjunction with the accompanying drawings in which:

FIG. 1 shows increased expression of MAP17 gene (A) and decreased expression of filaggrin gene (B) in human keratinocytes by interleukins derived from T helper 1 (Th1), T helper 2 (Th2) and T helper 17 (Th17) cells, as compared with the non-treated control group;

FIG. 2 schematically shows a cloning MAP17 gene (SEQ ID NO: 3);

FIG. 3 schematically shows a full length MAP17 of 589 nucleotides (SEQ ID NO: 4), of which carboxy-terminal (C-terminal) fragment of MAP17 is also identified;

FIG. 4 shows expression of MAP17 gene (A) and filaggrin gene (B) when MAP17 is overexpressed in human keratinocytes (HaCaT cell line); and

FIG. 5 shows a result of reverse transcription polymerase chain reaction (RT-PCR) for expression of MAP17 gene (A) and filaggrin gene (B) in normal human keratinocytes treated with or without ginsenoside-Re.

### DETAILED DESCRIPTION

Exemplary embodiments now will be described more fully hereinafter with reference to the accompanying drawings, in which exemplary embodiments are shown. This disclosure may, however, be embodied in many different forms and should not be construed as limited to the exemplary embodiments set forth therein. Rather, these exemplary embodiments are provided so that this disclosure will be thorough and complete, and will fully convey the scope of this disclosure to those skilled in the art. In the description, details of well-known features and techniques may be omitted to avoid unnecessarily obscuring the presented embodiments.

The terminology used herein is for the purpose of describing particular embodiments only and is not intended to be limiting of this disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item. The use of the terms “first”, “second”, and the like does not imply any particular order, but they are included to identify individual elements. Moreover, the use of the terms first, second, etc. does not denote any order or importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

A method for screening a material for improving skin functions according to the disclosure includes: (a) treating a skin cell with a candidate material; (b) detecting a change in a relative expression level of membrane-associated protein 17 (MAP17) gene; and (c) selecting a candidate material inducing the change in the expression level of the gene as a material for improving skin functions.

In (a), a skin cell is treated with a candidate material. In order to evaluate the candidate material's effect of defense from external stimulations, prevention of moisture evaporation, and maintenance of skin barrier function, the skin cell may be, for example, a human epidermal neonatal keratinocyte cell.

In (b), it is detected whether a relative expression level of MAP17 gene is changed due to the candidate material.

The relative expression level refers to the expression level of the gene in a candidate material-treated skin cell group as compared to an untreated skin cell group. In the whole description, the expression level may mean the relative expression level of the gene.

The inventors have ascertained that a material for improving skin functions may be screened by measuring the change of an expression level of a gene, particularly the MAP17 gene, as a marker.

The expression level of the MAP17 gene may be increased, for example, by one or more interleukin(s) selected from a group consisting of interferon-gamma (IFN-γ) derived from T helper 1 (Th1) cell, interleukin-4 (IL-4) derived from T helper 2 (Th2) cell, interleukin-17A (IL-17A) derived from T helper 17 (Th17) cell, interleukin-17F (IL-17F), interleukin-22 (IL-22) and interleukin-6 (IL-6).

As demonstrated through the following examples, the inventors have ascertained that the expression of the MAP17 gene increases remarkably by the interleukins derived from Th1 cell, Th2 cell and Th17 cell, which are involved in the decrease of skin moisturizing factors, damage of skin barrier function, decreased skin defense ability, and inflammation responses.

Through this, it was verified that the MAP17 gene is regulated by the interleukins which are increased by skin inflammations or skin barrier damages. It was further confirmed that the MAP17 gene may be a protein playing an important role in skin inflammations or skin barrier damages.

Accordingly, a significant decrease of the expression level of the MAP17 gene in a skin cell treated with a candidate material may indicate that the candidate material may be effectively utilized for improving skin barrier function, promoting skin moisturization, or preventing skin aging. Therefore, a detection of a change in the expression level of the MAP17 gene may be useful for screening a material for improving skin functions.

In another embodiment, filaggrin gene may also be used as a marker. In that case, a method for screening may further include detecting a change in an expression level of the filaggrin gene. When a skin cell is treated with a candidate material, the expression level of the filaggrin gene may increase as compared with an untreated skin cell group.

The increase of the expression level of the filaggrin gene may have a significant effect on the improvement of skin barrier function. The filaggrin protein may be degraded into several hydrophilic amino acids through a post-transcriptional modification process. The resultant amino acid pool constitutes natural moisturizing factors (NMFs), which help maintaining the stratum corneum moisturized. However, it was recently found out that the mutation of the filaggrin gene may result in decrease of skin moisturizing factors, damage of skin barrier function, decrease of skin defense ability, and acute or chronic inflammations through activation of T helper cells.

In this regard, the inventors first found out that the expression of the filaggrin gene is related with the expression level of the MAP17 gene, as demonstrated through the following examples. They ascertained that the expression level of the filaggrin gene may decrease if the expression of the MAP17 gene increases.

The structure of the MAP17 gene is as follows. A membrane binding site is present at the amino terminal (N-terminal), and a PDZ [post synaptic density protein (PSD95), Drosophila disc large tumor suppressor (DlgA), and zonula occludens-1 protein (zo-1)] domain binding site is present at the carboxy terminal (C-terminal). The PDZ domain binding site at the C-terminal of MAP17 provides the possibility of various protein-protein interactions, and signal transduction is possible therethrough.

In particular, the inventors have ascertained that the expression of the MAP17 gene may be further increased when the C-terminal fragment of the MAP17 gene is overexpressed as compared with when full length MAP17 is overexpressed, and that the expression level of the filaggrin gene may be significantly decreased accordingly.

Since the expression level of the filaggrin gene, which is involved in the improvement of skin barrier function, can be regulated directly and/or indirectly through the expression of the MAP17 gene, it was verified that the regulation of the expression level of the MAP17 gene is also closely related with the improvement of skin barrier function and that the regulation of the expression level may be utilized to effectively screen a material for improving skin functions.

In (c), the candidate material inducing the change in the expression level of the MAP17 gene is selected as a material for improving skin functions.

The inventors have ascertained the relationship between the change in the expression level of the MAP17 gene and the improvement of skin functions, and selected the MAP17 gene as a marker that can be used for screening a material for improving skin functions. In (c), for example, if the skin cell is treated with a candidate material, the candidate material that decreases the expression level of the MAP17 gene may be selected as a material for improving skin functions.

The improvement of skin functions may refer to, for example, improvement in skin barrier function, promotion of skin moisturization, prevention of skin aging, or amelioration of skin troubles, by means of decreasing the expression level of the MAP17 gene.

### EXAMPLES

The examples will now be described. The following examples are for illustrative purposes only and not intended to limit the scope of this disclosure.

### Example 1

### Change in Expression of MAP17 Gene by Inflammation-Related Interleukins

Human keratinocytes (human epidermal neonatal keratinocyte cells) were purchased from Lonza, Inc. (Walkersville, Md., USA) and subcultured according to the manufacturer's recommendations. The cells were incubated in a CO₂ incubator under a condition of 37° C. and 5% CO₂. Cell culture was prepared according to the instructions of Lonza, Inc. KGM-2 bullet kit [bovine pituitary extract (BPE, 2 mL), human epidermal growth factor (hEGF, 0.5 mL), insulin (0.5 mL), hydrocortisone (0.5 mL), transferrin (0.5 mL), epinephrine (0.5 mL), gentamycin sulfate+amphotericin B (GA-1000, 0.5 mL)] was added to KBM-2 (Clonetics CC-3103) medium (500 mL).

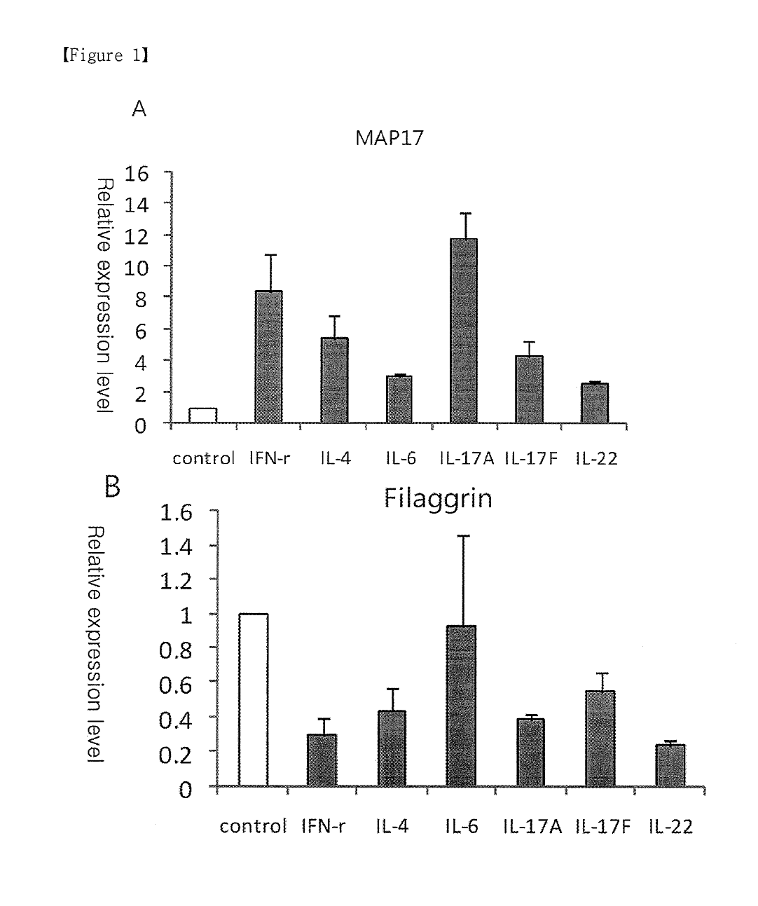

The cultured human keratinocytes without any treatment were used as a control group. For test groups, the human keratinocytes were further cultured for 24 hours after adding IFN-γ (200 units/mL), or IL-4, IL-6, IL-17A, IL-17F or IL-22 (50 ng/mL). The interleukins derived from different T helper (Th) cells were purchased from R&D Systems (Minneapolis, Minn., USA) and prepared into solutions according to the manufacturer's instructions. 24 hours after the interleukin treatment, the cells were washed twice with 10 mL of phosphate buffered saline (PBS) and total RNA was isolated from the cells using Trizol reagent (Invitrogen, Carlsbad, Calif., USA). The isolated RNA was purified once more using Qiagen RNeasy kit (Qiagen, Valencia, Calif.) and RNA quality was verified using Agilent 2100 BioAnalyzer (Agilent Technologies, Santa Clara, Calif., USA). cDNA was synthesized from the isolated RNA using Superscript Reverse Transcriptase (RT) II kit (Invitrogen, Carlsbad, Calif.), and expression of membrane-associated protein 17 (MAP17) and filaggrin genes was quantitatively analyzed by means of real time-reverse transcription polymerase chain reaction (Q-RT-PCR). Change in the expression pattern of MAP17 (Hs00173779_m1: SEQ ID NO: 1) and filaggrin (Hs00856927_g1: SEQ ID NO: 2), which is a differentiation marker gene of human keratinocyte, was evaluated using TaqMan® gene expression assay kit (Applied Biosystems, Foster City, Calif.) and is shown in FIG. 1.

As seen from FIG. 1, the interleukins derived from Th1, Th2 and Th17 cells markedly increase the expression of the MAP17 gene (A). In contrast, they inhibit the expression of the filaggrin gene (B).

### Example 2

### Quantification of Expression of Filaggrin Gene Due to Overexpression of MAP17

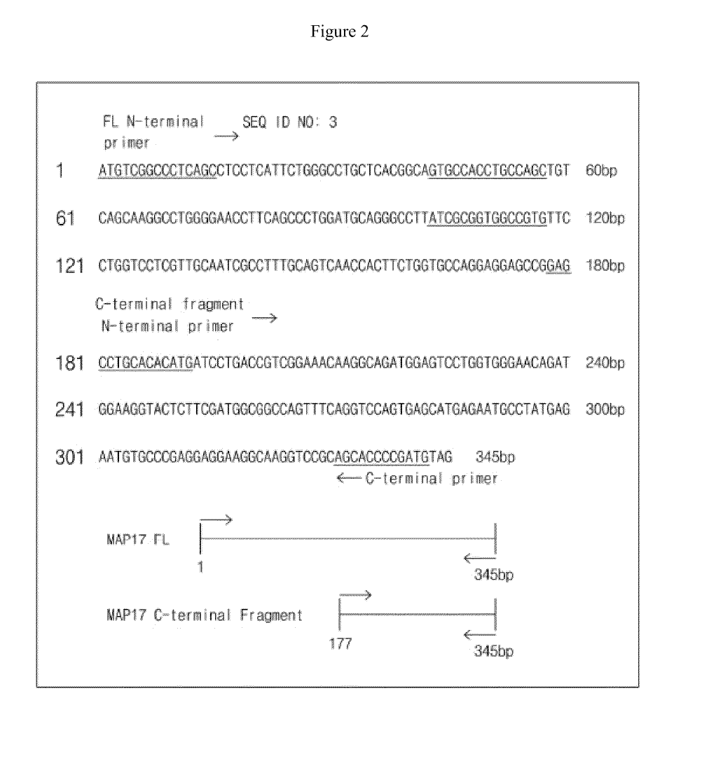

Clones having MAP17 gene (Clone id: hmu001988) were purchased from Korea UniGene (21C Human Gene Bank, Genome Research Center, KRIBB, Daejeon, Korea). In order to express the MAP17 gene in mammalian cells, full length MAP17 (1-345 base pairs, 115 amino acids) and carboxy-terminal (C-terminal) fragment (177-345 base pairs, 55 amino acids) were amplified by polymerase chain reaction (PCR). The N-terminal primer of the full length gene was 5′-GAA GAA TTC ATG TCG GCC CTC AGC-3′, including the EcoR1 restriction enzyme site. And, the N-terminal primer of the C-terminal fragment was 5′-GAA GAA TTC GAG CCT GCA CAC ATG-3′, including the EcoR1 restriction enzyme site. The C-terminal primer of the full length MAP17 gene and the C-terminal fragment was 5′-GAA CTC GAG TTA CAT CGG GGT GCT-3′, including the XhoI restriction enzyme site. PCR mixture solution included 0.1 μg DNA template, 0.2 mM dNTP, 0.2 μM primers and 0.5 unit Taq DNA polymerase (Invitrogen, Carlsbad, Calif., USA) (FIG. 2).

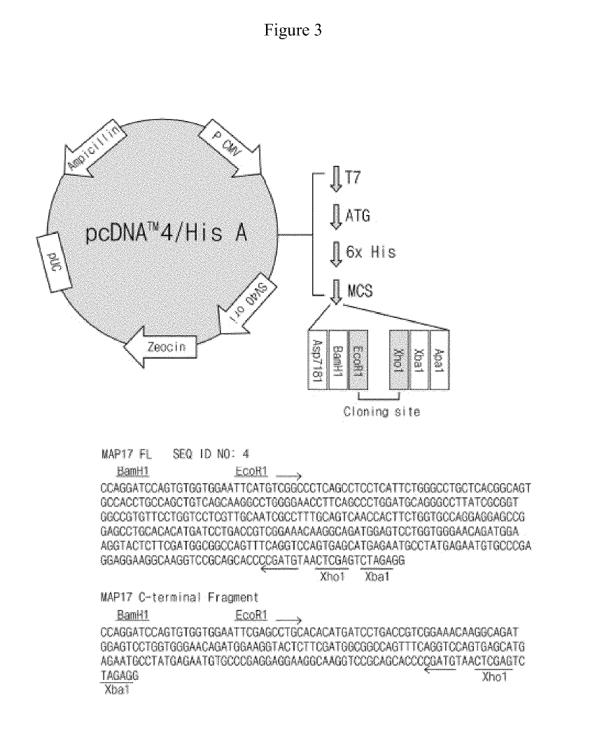

PCR condition was: 1 minute at 95° C.; 30 cycles of 1 minute at 95° C., 30 seconds at 55° C.; and 1 minute at 72° C.; followed by 5 minutes at 72° C. The PCR product was purified using QIAquik PCR purification kit (Qiagen, USA) and fragmented at 37° C. for 2 hours using pcDNA™4/His vector (Invitrogen, Carlsbad, Calif.) and EcoR1 and XhoI restriction enzymes. Plasmid ligation was carried out overnight at 16° C. using T4 DNA polymerase. Following the plasmid ligation, the product was transformed into DH5α cells, and the cells were grown in Luria-Bertani (LB) agar medium containing 50 μg mL⁻¹ ampicillin. Newly recombined plasmid was prepared from the grown cell colony, and the result was confirmed by DNA sequencing (FIG. 3).

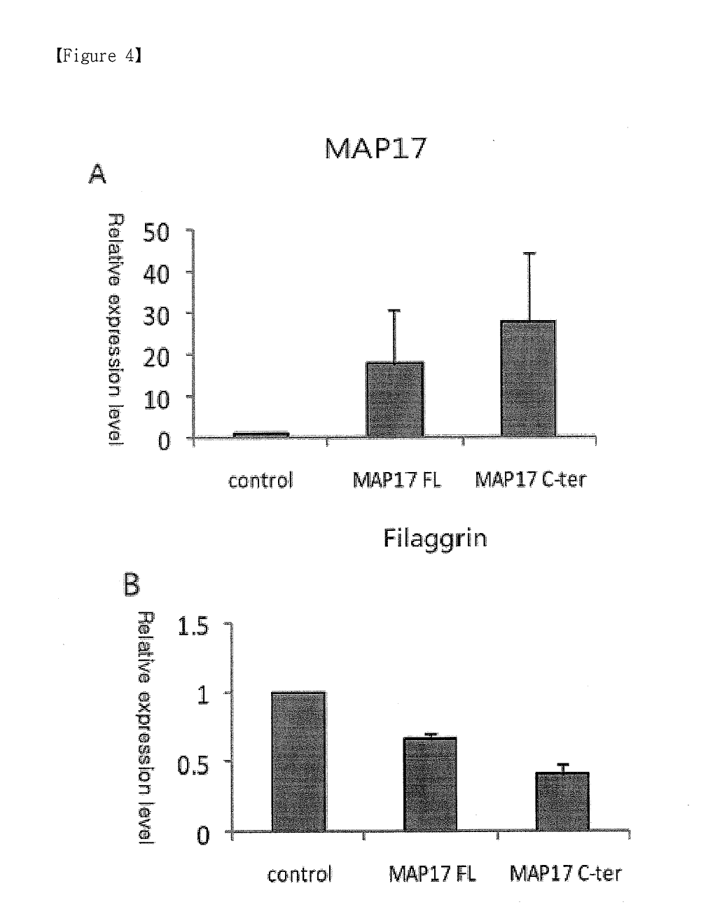

Human keratinocytes (HaCaT cell lines, acquired from Dr. N. E. Fusenig, Deutsches Krebsforschungszentrum, Heidelberg, Germany) were cultured in Dulbecco's modified Eagle's medium (DMEM) containing 10% fetal bovine serum (FBS) and 1% antibiotics (Cambrex, Walkersville, Md., USA) under a condition of 37° C. and 5% CO₂. The HaCaT cells were cultured on a 6-well plate, at 2×10⁴ cells/cm². 24 hours later, 1 ug of MAP17 full length plasmid and C-terminal fragment plasmid were transfected using FuGENE6 transfection reagent. 24 hours later, cell culture medium was replaced. 72 hours after the transfection, the cells were washed twice with 10 mL of PBS and total RNA was isolated from the cells using Trizol reagent (Invitrogen, Carlsbad, Calif., USA). The isolated RNA was purified once more using Qiagen RNeasy kit (Qiagen, Valencia, Calif.) and RNA quality was verified using Agilent 2100 BioAnalyzer (Agilent Technologies, Santa Clara, Calif., USA). cDNA was synthesized from the isolated RNA using Superscript RT II kit (Invitrogen, Carlsbad, Calif.) and was quantitatively analyzed by means of Q-RT-PCR. Change in the expression pattern of MAP17 (Hs00173779_m1) and filaggrin (Hs00856927_g1), which is a differentiation marker gene of human keratinocyte, was evaluated using TaqMan® gene expression assay kit (Applied Biosystems, Foster City, Calif.) and is shown in FIG. 4.

As seen from FIG. 4, the expression of the MAP17 gene was markedly increased when the full length MAP17 and the C-terminal fragment were overexpressed (A). In contrast, the expression of the filaggrin gene was significantly decreased when the MAP17 C-terminal fragment was overexpressed as compared with when the full length MAP17 was overexpressed (B).

### Example 3

### Confirmation of Regulation of Expression Level of MAP17 and Filaggrin Genes by Ginsenoside-Re

Various human keratinocytes were treated with a variety of materials, and change in expression of MAP17 and filaggrin genes were monitored. It was investigated whether ginsenoside-Re, a kind of ginsenoid derived from the fruit of ginseng, regulates the expression of MAP17 and filaggrin genes. Human keratinocytes were purchased from Lonza, Inc. and cultured in KBM-2 medium (Clonetics CC-3103) in a CO₂ incubator under a condition of 37° C. and 5% CO₂.

The cultured human keratinocytes without any treatment were used as a control group. For a test group, the human keratinocytes were further cultured for 24 hours after adding ginsenoside-Re (10 uM). Ginsenoside-Re was purchased from Wako (Kanagawa, Japan) and prepared into a solution according to the manufacturer's instructions. 24 hours after the ginsenoside-Re treatment, the cells were washed twice with 10 mL of PBS and total RNA was isolated from the cells using Trizol reagent (Invitrogen, Carlsbad, Calif., USA).

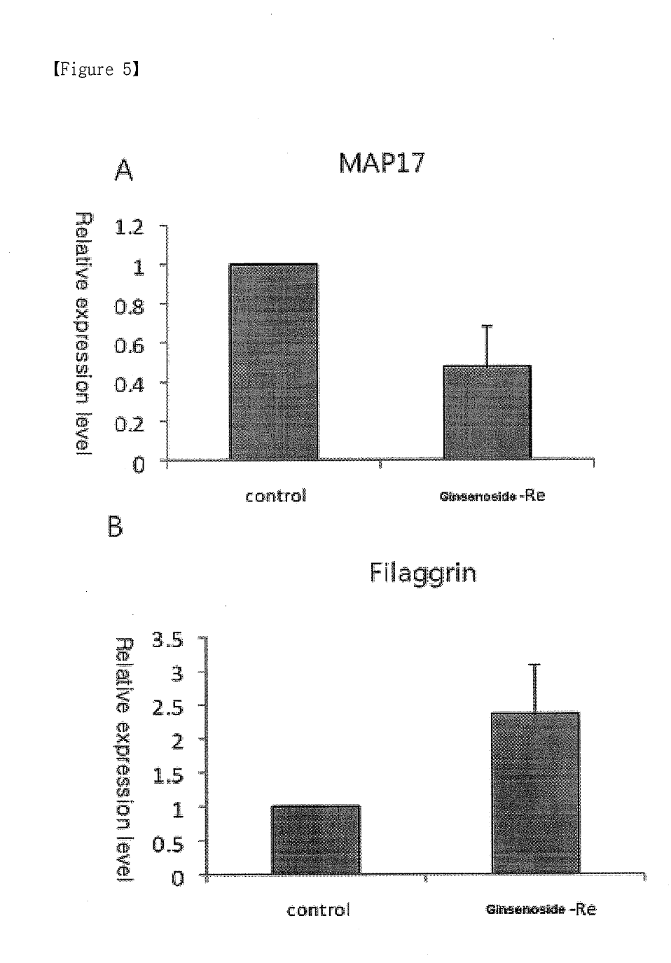

The isolated RNA was purified once more using Qiagen RNeasy kit (Qiagen, Valencia, Calif.) and RNA quality and concentration were verified using Agilent 2100 BioAnalyzer (Agilent Technologies, Santa Clara, Calif., USA). cDNA was synthesized from the isolated RNA using Superscript RT II kit (Invitrogen, Carlsbad, Calif.), and change in gene expression was quantitatively analyzed by means of Q-RT-PCR. Change in the expression pattern of MAP17 (Hs00173779_m1) and filaggrin (Hs00856927_g1) was evaluated using TaqMan® gene expression assay kit (Applied Biosystems, Foster City, Calif.) and is shown in FIG. 5.

As seen from FIG. 5, ginsenoside-Re markedly decreases the expression of the MAP17 gene in human keratinocytes (A). In contrast, it increases the expression of the filaggrin gene in human keratinocytes (B).

While the exemplary embodiments have been shown and described, it will be understood by those skilled in the art that various changes in form and details may be made thereto without departing from the spirit and scope of this disclosure as defined by the appended claims.

In addition, many modifications can be made to adapt a particular situation or material to the teachings of this disclosure without departing from the essential scope thereof. Therefore, it is intended that this disclosure not be limited to the particular exemplary embodiments disclosed as the best mode contemplated for carrying out this disclosure, but that this disclosure will include all embodiments falling within the scope of the appended claims.

What is claimed is:

1. A method of screening a material for improving skin functions, comprising:
treating a skin cell with a candidate material;
detecting a change in a relative expression level of membrane-associated protein 17 (MAP17) gene; and
selecting a candidate material inducing the change in the expression level of the gene as a material for improving skin functions.

2. The method of screening a material for improving skin functions according to claim 1, wherein the skin cell is a human keratinocyte.

3. The method of screening a material for improving skin functions according to claim 1, wherein, in said selecting the material for improving skin functions, a candidate material which decreases the expression level of the MAP17 gene is selected as a material for improving skin functions.

4. The method of screening a material for improving skin functions according to claim 1, wherein the material for improving skin functions improves skin barrier function, promotes skin moisturization, prevents skin aging, or ameliorates skin troubles.


SEQUENCE LISTING (4 sequences)

SEQ ID NO: 1 - DNA, 894 bp, Homo sapiens
gcccgtcttcgtgtctcctccctccctcgccttcctccttcctagctcctctcctccagggccagactgagcccaggttgatttcaggcggacaccaatagactccacagcagctccaggagcccagacaccggcggccagaagcaaggctaggagctgctgcagccatgtcggccctcagcctcctcattctgggcctgctcacggcagtgccacctgccagctgtcagcaaggcctggggaaccttcagccctggatgcagggccttatcgcggtggccgtgttcctggtcctcgttgcaatcgcctttgcagtcaaccacttctggtgccaggaggagccggagcctgcacacatgatcctgaccgtcggaaacaaggcagatggagtcctggtgggaacagatggaaggtactcttcgatggcggccagtttcaggtccagtgagcatgagaatgcctatgagaatgtgcccgaggaggaaggcaaggtccgcagcaccccgatgtaaccttctctgtggctccaaccccaagactcccaggcacatgggatggatgtccagtgctaccacccaagccccctccttctttgtgtggaatctgcaatagtgggctgactccctccagccccatgccggccctacccgcccttgaagtatagccagccaaggttggagctcagaccgtgtctaggttggggctcggctgtggccctggggtctcctgctcagctcagaagagccttctggagaggacagtcagctgagcacctcccatcctgctcacacgtccttccccataactatggaaatggccctaatttctgtgaaataaagactttttgtatttctggggctgaggctcagcaacagcccctcaggcttccagtga

SEQ ID NO: 2 - DNA, 12747 bp, Homo sapiens
cttttggtgaacaaggttcacatttattgccaaaagatgtctactctcctggaaaacatctttgccataattaatcttttcaagcaatattcaaaaaaagataaaaacactgacacattgagtaaaaaagagctgaaggaacttctggaaaaggaatttcggcaaatcctgaagaatccagatgacccagatatggttgatgtcttcatggatcacttggatatagaccacaacaagaaaattgacttcactgagtttcttctgatggtattcaagttggctcaagcatattatgagtctaccagaaaagagaatttaccgatatcaggacacaagcacagaaagcacagtcatcatgataaacatgaagataataaacaggaagaaaacaaagaaaacagaaaaagaccctcaagtctggaaagaagaaacaatagaaaagggaataagggaagatccaagagcccaagagaaacaggggggaaaaggcatgaatctagttctgaaaaaaaagaaagaaaaggatattcacctactcatagagaagaagaatatggaaaaaaccatcataactcaagtaaaaaagagaaaaacaagactgaaaatactagattaggagacaataggaagaggctaagtgaaagacttgaagagaaagaagacaatgaagaaggagtatatgattatgaaaatacaggaagaatgactcaaaaatggatacaatcaggccatattgccacatattacacaatccaggatgaagcctatgacaccactgatagtctattagaagaaaacaaaatatatgaaagatcaaggtcatctgatggcaaatcatcatctcaagtgaacaggtcaagacatgaaaatacaagccaggtaccattgcaggagtccaggacaagaaagcgtaggggatccagagttagccaggacagggacagtgagggacactcagaagactctgagaggcactctgggtcggcttccagaaaccatcatggatctgcgtgggagcagtcaagagatggctccagacaccccaggtcccatgatgaagacagagccagtcatgggcactctgcagacagctccagacaatcaggcactcgtcacgcagagacttcctctcgtggacagactgcatcatcccatgaacaggcaagatcaagtccaggagaaagacatggatccggccaccagcagtcagcagacagctccagacactcagccactgggcgcgggcaagcttcatctgcagtcagcgatcgtggacaccgggggtctagcggtagtcaggccagtgacagtgagggacattcagaaaactcagacacacaatcagtgtcaggccacggaaaggctgggctgagacagcagagccaccaagagtccacacgtggccggtcaggggaacggtctggacgttcagggtcttccctctaccaggtgagcactcatgaacagcctgactctgcccatggacggaccgggaccagcactggaggaagacaaggatcgcaccacgagcaggcacgagacagctccaggcattcagcgtcccaagagggtcaggacaccattcgtggacacccggggtcaagcagaggaggaaggcagggatcccaccacgagcaatcggtaaataggtctggacactcaggttcccatcacagccacaccacatcccagggaaggtctgatgcctcccatgggcagtcaggatccagaagtgcaagcagacaaacacgaaatgaggaacaatcaggagacggcaccaggcactcagggtcacgtcatcatgaagcttcctctcaggctgacagctctagacactcacaggtgggccagggacaatcatcggggcccaggacaagtaggaaccagggatccagtgttagccaggacagtgacagtcagggacactcagaagactctgagaggtggtctgggtctgcttccagaaaccatcatggatctgctcaggagcagtcaagagatggctccagacaccccaggtcccatcacgaagacagagctggtcatgggcactctgcagacagctccagaaaatcaggcactcgtcacacacagaattcctctagtggacaggctgcgtcatcccatgaacaggcaagatcaagtgcaggagaaagacatggatcccgccaccagctccagtcagcagacagctccagacactcaggcactgggcacggacaagcttcatctgcagtcagagacagtggacaccgagggtccagtggtagtcaggccactgacagtgagggacattcagaagactcagacacacagtcagtgtcaggccatggacaggctggtcaccatcagcagagccaccaagagtccgcacgtgaccggtcaggggaaaggtctcgacgttcagggtctttcctctaccaggtgagcactcataaacagtctgagtcctcccatggatggacagggcccagcactggagtaagacaaggatcccaccatgagcaggcacgagacaactccaggcactcagcatcccaagatggtcaggacaccattcgtggacacccggggtcaagcagaagaggaaggcaggggtcccaccacgagcaatcggtagataggtctggacactcagggtcccatcacagccacaccacatcccagggaaggtctgatgcctcccgtgggcagtcaggatccagaagtgcaagcagaacaacacgtaatgaggaacaatcaagagacggctccaggcactcagggtcacgtcaccatgaagcttcctctcatgccgacatctctagacactcacaggcaggccagggacaatcagaggggtccaggacaagcaggcgccagggatccagtgttagccaggacagtgacagtgagggacattcagaagactctgagaggtggtctgggtctgcttccagaaaccatcgtggatctgctcaggagcagtcaagacatggctccagacaccccaggtcccatcacgaagacagagccggtcacgggcactctgcagacagctccagacaatcaggaactcctcacgcagagacttcctctggtggacaggctgcgtcatcccatgaacaggcaagatcaagtccaggagaaagacacggatcccgccaccagcagtcagcagacagctccagacactcaggcattccgcgcagacaagcttcatctgcagtcagagacagtggacactgggggtccagtggtagtcaggccagtgatagtgagggacattcagaggagtcagacacacagtcagtgtcaggccatggacaggatgggccccatcagcagagccaccaagagtccgcacgtgactggtcagggggaaggtctggacgttcagggtctttcatctaccaggtgagcactcatgaacagtctgagtctgcccatgggcggaccaggaccagcactggacgaagacaaggatcccaccacgagcaggcacgagacagctccaggcactcagcgtcccaagagggtcaggacaccattcgtgcacacccggggtcaaggagaggaggaaggcagggatcccaccatgagcaatcggtagatagatctggacactcagggtcccatcacagccacaccacatcccagggaaggtctgatgcctcccatgggcagtcaggatccagaagtgcaagcagacaaactcgtaaggacaaacaatcaggagacggctccaggcactcagggtcacgtcaccatgaagctgcctcttgggctgacagctctagacactcacaggtgggacaggaacaatcatcggggtccaggacaagcaggcaccagggatccagtgttagccaggacagtgacagtgagagacactcagacgactccgagaggttgtctgggtctgcttccagaaaccatcatggatcttctcgggagcagtcaagagatggctccagacaccctgggttccatcaagaagacagagccagtcacgggcactctgcagacagctccagacaatcaggcactcatcacacagagtcttcctctcatggacaggctgtgtcatcccatgaacaggcaagatcaagtccaggagaaagacatggatcccgccaccagcagtcagcagacagctccagacactcaggcattgggcacagacaagcttcatctgcagtcagagacagtggacaccgagggtccagtggtagtcaggtcactaacagtgagggacattcagaagactcagacacacagtcagtgtcagcccacggacaagctgggccccatcagcagagccacaaagagtccgcacgtggccagtcaggggaaagctctggacgttcaaggtctttcctctaccaggtgagctctcatgaacagtctgagtccacacacggacagactgcacccagcactggaggaagacaaggatcccgccatgagcaggcacgaaacagctctaggcactcagcatcccaagacggtcaggacaccattcgtggacacccggggtcaagcagaggaggaaggcagggatcctaccacgagcaatcagtagataggtctggacactcagggtaccatcacagccacaccacaccccagggaaggtctgatgcctcccatgggcagtcaggacccagaagtgcaagcaggcaaacaagaaatgaggaacaatcaggagacggctccaggcactcagggtcacgtcaccatgaaccttccactcgggccggcagctctagacactcacaggtgggccagggagaatcagcggggtccaagacaagcaggcgccagggatccagtgttagtcaggacagggacagtgagggacactcagaagactctgagaggcggtctgagtcggcttccagaaaccattatggatctgctcgggagcagtcaagacatggctccaggaaccccaggtcccatcaagaagatagagccagtcatgggcactctgcagagagctccagacaatcaggcactcgtcatgcagagacttcctctggtggacaggctgcatcatcccaggaacaggcaaggtcaagtccaggagaaagacatggatcccgccaccagcagtcagcagacagctccacagactcaggcactgggcgcagacaagattcatctgtagtcggagacagtggaaaccgagggtccagtggtagccaggccagtgacagcgagggacactcagaagagtcagacacacagtcagtgtcagcccacggacaggctgggccccatcagcagagccaccaagagtccacacgtggccagtcaggggaaaggtctggacgttcagggtctttcctctaccaggtgagcactcatgaacagtctgagtccgcccatggacgcacagggcccagcactggaggaagacaaagatcccgccacgagcaggcacgagacagctccaggcactcagcgtcccaagagggtcaggacaccattcgtggacacccagggtcaagcagaggaggaaggcagggatcccactatgagcaatcggtagatagttctggacactcagggtctcatcacagccacaccacgtcccaggaaaggtctgatgtctcccgtgggcagtcaggatccagaagtgtcagcagacaaacacgtaatgagaaacaatcaggagacggctccaggcactcagggtcgcgtcaccatgaagcttcctctcgggccgacagctctagacactcgcaggtgggccagggacaatcatcagggcccaggacaagcaggaaccagggatccagtgttagccaggacagtgacagtcagggacactcagaagactctgagaggtggtctgggtctgcttccagaaaccatcttggatctgcttgggagcagtcaagagatggctccagacaccctgggtcccatcacgaagacagagccggtcacgggcactctgcagacagctccagacaatcaggcactcgtcacacagagtcttcctctcgtggacaggctgcgtcatcccatgaacaggcaagatcaagtgcaggagaaagacatggatcccaccaccagctccagtcagcagacagctccagacactcaggcattgggcatggacaagcttcatctgcagtcagagacagtggacaccgagggtacagtggtagtcaggccagtgacagtgagggacattcagaagactcagacacacagtcagtgtcagcacagggaaaagctgggccccatcagcagagccacaaagagtccgcacgtggccagtcaggggaaagctctggacgttcagggtctttcctctaccaggtgagcactcatgaacagtctgagtccacccatggacagtctgcgcccagcactggaggaagacaaggatcccattatgatcaggcacaagacagctccaggcactcagcatcccaagagggtcaggacaccattcgtggacacccggggccaagcagaggaggaagacaggggtcccaccaagagcaatcggtagataggtctggacactcagggtctcatcacagccacaccacatcccagggaaggtctgatgcctcccgtgggcagtcaggatccagaagtgcaagcagaaaaacatatgacaaggaacaatcaggagatggctctaggcactcagggtcgcatcatcatgaagcttcctcttgggccgacagctctagacactcactggtgggccagggacaatcatcagggcccaggacaagcaggccccggggatccagtgttagccaggacagtgacagtgagggacactcagaagattctgagaggcggtctgggtctgcgtccagaaaccatcatggatctgctcaggagcagtcaagagatggctccagacaccccaggtcccatcacgaagacagagccggtcatgggcactctgcagagagctccagacaatcaggcactcatcatgcagagaattcctctggtggacaggctgcatcatcccatgaacaggcaagatcaagtgcaggagagagacacggatcccaccaccagcagtcagcagacagctccagacactcaggcattgggcacggacaagcttcatctgcagtcagagacagtggacaccgagggtccagtggtagtcaggccagtgacagtgagggacattcagaagactcagacacacagtcagtgtcagcccacggacaggctgggccccatcagcagagccaccaagagtccacacgtggccggtcagcaggaaggtctggacgttcagggtctttcctctaccaggtgagcactcatgaacagtctgagtccgcccatggacggaccgggaccagcactggaggaagacaaggatcccaccacaagcaggcacgagacagctccaggcactcaacgtcccaagagggtcaggacaccattcatggacacccggggtcaagcagtggaggaaggcagggatcccactacgagcaattggtagatagatctggacactcagggtctcatcacagccacaccacatcccagggaaggtctgatgcctcccatgggcactcaggatccagaagtgcaagcagacaaactcgtaacgatgaacaatcaggagacggctccaggcactcagggtcgcgtcaccatgaagcttcctctcgggccgacagctctggacactcgcaggtgggccagggacaatcagaggggcccaggacaagcaggaactggggatccagttttagccaggacagtgacagtcagggacactcagaagactctgagaggtggtctgggtctgcttccagaaaccatcatggatctgctcaggagcagctaagagatggctccagacaccccaggtcccatcaagaagacagagctggtcatgggcactctgcagacagctccagacaatcaggcactcgtcacacacagacttcctctggtggacaggctgcatcatcccatgaacaggcaagatcaagtgcaggagaaagacatggatcccaccaccagcagtcagcagacagctccagacactcaggcattgggcacggacaagcttcatctgcagtcagagacagtggacaccgagggtacagtggtagtcaggccagtgacaatgagggacattcagaagactcagacacacagtcagtgtcagcccacggacaggctgggtcccatcagcagagccaccaagagtccgcacgtggccggtcaggggaaacgtctggacattcaggatctttcctctaccaggtgagcactcatgaacagtctgagtcctcccatggatggacggggcccagcactagaggaagacaaggatcccgccatgagcaggcacaagacagctccaggcactcagcatcccaagacggtcaggacaccattcgtggacacccggggtcaagcagaggaggaaggcaggggtaccaccacgagcattcggtagatagctctggacactcagggtcccatcacagccacaccacatcccagggaaggtctgatgcctcccgtgggcagtcaggatccagaagtgcaagcagaacaacacgtaatgaggaacaatcaggagacggctccaggcactcagggtcgcgtcaccatgaagcttccactcatgccgacatctctagacactcacaggcagtccagggacaatcagaggggtccaggagaagcaggcgccagggatccagtgtgagccaggacagtgacagtgagggacattcagaagactctgagaggtggtctgggtctgcttccagaaaccatcatggatctgctcaggagcagctaagagatggctccagacaccccaggtcccatcaagaagacagagctggtcatgggcactctgcagacagctccagacaatcaggcactcgtcacacacagacttcctctggtggacaggctgcatcatcccatgaacaggcaagatcaagtgcaggagaaagacatggatcccaccaccagcagtcagcagacagctccagacactcaggcattgggcacggacaagcttcatctgcagtcagagacagtggacaccgagggtacagtggtagtcaggccagtgacaatgagggacattcagaagactcagacacacagtcagtgtcagcccacggacaggctgggtcccatcagcagagccaccaagagtccgcacgtggccggtcaggggaaacgtctggacattcaggatctttcctctaccaggtgagcactcatgaacagtctgagtcctcccatggatggacggggcccagcactagaggaagacaaggatcccgccatgagcaggcacaagacagctccaggcactcagcatcccaatacggtcaggacaccattcgtggacacccggggtcaagcagaggaggaaggcaggggtaccaccacgagcattcggtagatagctctggacactcagggtcccatcacagccacaccacatcccagggaaggtctgatgcctcccgtgggcagtcaggatccagaagtgcaagcagaacaacacgtaatgaggaacaatcaggagacagctccaggcactcagtgtcacgtcaccatgaagcttccactcatgccgacatctctagacactcacaggcagtccagggacaatcagaggggtccaggagaagcaggcgccagggatccagtgtgagccaggacagtgacagtgagggacattcagaagactctgagaggtggtctgggtctgcttccagaaaccatcgtggatctgttcaggagcagtcaaggcacggctccagacaccccaggtcccatcacgaagacagagccggtcacgggcactctgcagaccgctccagacaatcaggcactcgtcacgcagagacttcctctggtggacaggctgcatcatcccatgaacaggcaagatcaagtccaggagagagacacggatcccgccaccagcagtcagcagacagctccagacactcaggcattccgcgtggacaagcttcatctgcagtcagagacagtagacactgggggtccagtggtagtcaggccagtgatagtgagggacattcagaagagtcagacacacagtcagtgtcaggccatggacaggctgggccccatcagcagagccaccaagagtccgcacgtgaccggtcagggggaaggtctggacgttcagggtctttcctctaccaggtgagcactcatgaacagtctgagtctgcccatgggcggaccaggaccagcactggacgaagacaaggatcccaccacgagcaggcacgagacagctccaggcactcagcgtcccaagagggtcaggacaccattcgtggacacccggggtcaagcagaagaggaaggcagggatcccactacgagcaatcggtagataggtctggacactcagggtcccatcacagccacaccacatcccagggaaggtctgatgcctcccgtgggcagtcaggatccagaagtgccagcagacaaactcgtaatgacgaacaatcaggagatggctccaggcactcatggtcgcatcaccatgaagcttccactcaggcggacagctctagacactcacagtccggccagggacaatcagcggggcccaggacaagcaggaaccagggatccagtgttagccaggacagtgacagtcagggacactcagaagactctgagaggtggtctgggtctgcttccagaaaccatcgtggatctgctcaggagcagtcaagagatggctccagacaccccacgtcccatcacgaagacagagccggtcacgggcactctgcagagagctccagacaatcaggcactcatcatgcagagaattcctctggtggacaggctgcatcatcccatgaacaggcaagatcaagtgcaggagagagacatggatcccaccaccagcagtcagcagacagctccagacactcaggcattgggcacggacaagcttcatctgcagtcagagacagtggacaccgagggtccagtggtagtcaggccagtgacagtgagggacattcagaagactcagacacacagtcagtgtcagcccacggacaggctgggccccatcagcagagccaccaagagtccacacgtggccggtcagcaggaaggtctggacgttcagggtctttcctctaccaggtgagcactcatgaacagtctgagtctgcccatggacgggctgggcccagtactggaggaagacaaggatcccgccacgagcaggcacgagacagctccaggcactcagcgtcccaagagggtcaggacaccattcgtggacacccggggtcaaggagaggaggaagacagggatcctaccacgagcaatcggtagataggtctggacactcagggtcccatcacagccacaccacatcccagggaaggtctgatgcctcccatgggcagtcaggatccagaagtgcaagcagagaaacacgtaatgaggaacagtcaggagacggctccaggcactcagggtcgcgtcaccatgaagcttccactcaggctgacagctctagacactcacagtccggccagggtgaatcagcggggtccaggagaagcaggcgccagggatccagtgttagccaggacagtgacagtgaggcatacccagaggactctgagaggcgatctgagtctgcttccagaaaccatcatggatcttctcgggagcagtcaagagatggctccagacaccccggatcctctcaccgcgatacagccagtcatgtacagtcttcacctgtacagtcagactctagtaccgctaaggaacatggtcactttagtagtctttcacaagattctgcgtatcactcaggaatacagtcacgtggcagtcctcacagttctagttcttatcattatcaatctgagggcactgaaaggcaaaaaggtcaatcaggtttagtttggagacatggcagctatggtagtgcagattatgattatggtgaatccgggtttagacactctcagcacggaagtgttagttacaattccaatcctgttgttttcaaggaaagatctgatatctgtaaagcaagtgcgtttggtaaagatcatccaaggtattatgcaacgtatattaataaggacccaggtttatgtggccattctagtgatatatcgaaacaactgggatttagtcagtcacagagatactattactatgagtaagaaattaatggcaaaggaattaatccaagaatagaagaatgaagcaagttcactttcaatcaagaaacttcataatactttcagggaagttatcttttcctgtcaatctgtttaaaatatgctatagtatttcattagtttggtggtagcttatttttattgtgtaatgatctttaaacgctatatttcagaaatattaaatggaagaaatcaatatcatggagagctaactttagaaaactagctggagtattttaggagattctgggtcaagtaatgttttatgtttttgaaagtttaagttttagacactccccaaatttctaaattaatctttttcagaaatatcgaaggagccaaaaatataaaacagttctgtataccaaagtggctatatcaacatcagggctagcacatctttctctattatccttctattggaattctagtattctgtattcaaaaaatcatcttggacataattaatattatagtaagctgcatctaaattaaaaataaactatt

SEQ ID NO: 3 - DNA, 345 bp, Homo sapiens
atgtcggccctcagcctcctcattctgggcctgctcacggcagtgccacctgccagctgtcagcaaggcctggggaaccttcagccctggatgcagggccttatcgcggtggccgtgttcctggtcctcgttgcaatcgcctttgcagtcaaccacttctggtgccaggaggagccggagcctgcacacatgatcctgaccgtcggaaacaaggcagatggagtcctggtgggaacagatggaaggtactcttcgatggcggccagtttcaggtccagtgagcatgagaatgcctatgagaatgtgcccgaggaggaaggcaaggtccgcagcaccccgatgtag

SEQ ID NO: 4 - DNA, 589 bp, Homo sapiens
ccaggatccagtgtggtggaattcatgtcggccctcagcctcctcattctgggcctgctcacggcagtgccacctgccagctgtcagcaaggcctggggaaccttcagccctggatgcagggccttatcgcggtggccgtgttcctggtcctcgttgcaatcgcctttgcagtcaaccacttctggtgccaggaggagccggagcctgcacacatgatcctgaccgtcggaaacaaggcagatggagtcctggtgggaacagatggaaggtactcttcgatggcggccagtttcaggtccagtgagcatgagaatgcctatgagaatgtgcccgaggaggaaggcaaggtccgcagcaccccgatgtaactcgagtctagaggccaggatccagtgtggtggaattcgagcctgcacacatgatcctgaccgtcggaaacaaggcagatggagtcctggtgggaacagatggaaggtactcttcgatggcggccagtttcaggtccagtgagcatgagaatgcctatgagaatgtgcccgaggaggaaggcaaggtccgcagcaccccgatgtaactcgagtctagagg